(19) United States
(12) Patent Application Publication
(10) Pub. No.: US 2026/0004683 A1
(43) Pub. Date: Jan. 1, 2026

(54) MODULAR TRACHEOSTOMY TASK TRAINER

(71) Applicant: New York University, New York, NY (US)

(72) Inventor: Brian Kaufman, North Hills, NY (US)

(21) Appl. No.: 19/256,736

(22) Filed: Jul. 1, 2025

Related U.S. Application Data
(60) Provisional application No. 63/666,524, filed on Jul. 1, 2024.

(51) Int. Cl. G09B 23/34; A61M 16/04; G09B 23/30

(52) U.S. Cl. CPC G09B 23/34; G09B 23/303; A61M 16/0465

(57) ABSTRACT

A modular tracheostomy task trainer device comprising a base unit comprising at least one attachment point, and at least one module comprising an attachment point configured to removably attach to the at least one attachment point of the base unit. A tracheostomy training kit comprising the modular tracheostomy task trainer device.

### CROSS-REFERENCE TO RELATED APPLICATIONS

This application claims priority to U.S. Provisional Patent Application No. 63/666,524 filed on Jul. 1, 2024, the contents of which are incorporated by reference herein in its entirety.

### BACKGROUND

A study reported on all major complications of airway management that occurred over a 1-year period in all the National Health Service Hospitals in the United Kingdom (Cook T M, et al. Br J Anaesth 2011. 106(5): 632-642). Thirty-six events occurred in intensive care units with 61% of these events leading to death or permanent neurologic injury. Tracheostomy-related events were the most frequently occurring problem (50%). An expert review panel that reviewed each individual event believed that inadequate education/training were either causal or a contributory factor in 58% of the events. Studies in teaching hospitals have found that operator inexperience is associated with adverse effects during endotracheal intubation in the Intensive Care Unit (ICU) and operation room (Baker, P. A et al., Anaesthesia 2011.66 (supp 2): 101-111; Roux D et al. Crit Care Med 2014. 42(4): 886-895). Current methods utilized to educate healthcare providers on how to evaluate and manage tracheostomy-related emergencies include didactic lectures, case discussions, and high-fidelity human patient simulation. However, high-fidelity human patient simulation does not realistically recreate the emergencies. No currently available commercially produced task trainers for tracheostomy education may be used for either formative training sessions or summative assessment of the ability of healthcare providers to identify and manage the most common and/or significant tracheostomy emergencies.

Thus, there is a need in the art for a task trainer that allows educators to teach providers how to assess and manage most of the life-threatening events related to tracheostomy complications.

### SUMMARY

Aspects of the present disclosure relate to a modular tracheostomy task trainer device comprising a base unit comprising at least one attachment point, and at least one module comprising an attachment point configured to removably attach to the at least one attachment point of the base unit. In some embodiments, the device further comprises a plurality of modules, wherein each module of the plurality of modules are interchangeable.

In some embodiments, the base unit comprises a body having a cavity therein, the cavity configured to receive the at least one module. In some embodiments, the base unit is shaped to replicate the head and neck anatomy and comprises an anatomical feature selected from a mouth or a nose. In some embodiments, the base unit comprises an oral cavity fluidly connected to an upper air passage. In some embodiments, the upper air passage comprises structures configured to replicate one or more of a pharynx, a nasopharynx, an oropharynx, a hypopharynx, a larynx, or an epiglottis. In some embodiments, the at least one module comprises a tracheal passage and an esophageal passage, wherein each passage is fluidly connectable to the upper air passage. In some embodiments, the at least one module comprises an opening in the tracheal passage, the opening sized to fit a tracheostomy tube.

In some embodiments, the at least one module replicates a normal trachea. In some embodiments, the at least one module comprises an obstruction in the tracheal passage, wherein the obstruction may be configured to fully or partially obstruct the tracheal passage. In some embodiments, the at least one module comprises an obstruction in the opening in the tracheal passage, configured to prevent the insertion of a tracheostomy tube into the opening. In some embodiments, the at least one module comprises one or more inlets in the tracheal passage fluidly connected to a fluid supply, wherein the one or more inlets are configured to introduce one or more fluids to the tracheal passage. In some embodiments, the one or more fluids may be a blood simulant or a mucus simulant.

In some embodiments, the base unit and the at least one module are formed of materials configured to replicate soft tissue textures, muscle, cartilage, mucosal lining, or cilia. In some embodiments, the materials are selected from the group consisting of: plastics, acrylic, metals, metal alloys, fiberglass, rubber, silicone, silicone rubber, cast urethane rubber, latex, polyethylene, polylactic acid (PLA), polyurethane, polyester, polyester resin, polyvinyl alcohol (PVA), polyether ether ketone (PEEK), acrylonitrile butadiene styrene (ABS), polycarbonate, polypropylene, carbon fibers, thermoplastic elastomers, resins, gels, foam, and any combinations thereof. In some embodiments, the base unit comprises at least a first and a second attachment point. In some embodiments, the at least one module comprises at least a first and a second attachment point, configured to removably attach to the first and second attachment points of the base unit, respectively. In some embodiments, the device further comprises a seal between the at least one attachment point of the base unit and the at least one attachment point of the at least one module.

In some embodiments, the base unit and the at least one module are sized to replicate the head and neck anatomy of an average adult. In some embodiments, the base unit and the at least one module are sized to replicate the head and anatomy of a child between the ages of 0 and 12 years old. In some embodiments, the device is configured to simulate one or more of: tracheostomy tube insertion procedures, tracheostomy tube maintenance procedures, endotracheal tube insertion procedures, nasal intubation procedures, extubation procedures, laryngoscope insertion, visualization procedures, airway management techniques, suction, resuscitation techniques, and any combinations thereof. In some embodiments, the base unit and the at least one module are formed via injection molding, cast molding, or 3D printing.

Aspects of the present disclosure relate to a training kit for tracheostomy procedures, comprising a modular tracheostomy task trainer device, and one or more tracheostomy tubes, one or more endotracheal tubes, one or more resuscitator devices, one or more lung bags, one or more suction devices, one or more conduits, one or more pumps, one or more valves, a blood simulant, a blood simulant supply, a mucus simulant, a mucus simulant supply, or any combinations thereof. In some embodiments, the training kit further comprises one or more modular tracheostomy task trainer devices, each device being sized to simulate tracheostomy procedures on an average adult, or a child between the ages of 0 and 12 years old.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 depicts a perspective view of an exemplary modular task trainer device including an exemplary base unit and exemplary modules.

FIG. 2A depicts a front-view of an exemplary base unit of the modular task trainer device. FIG. 2B depicts a close-up view of an exemplary base unit. FIG. 2C depicts a close-up view of an exemplary base unit.

FIG. 3A depicts a top-down view of an exemplary base unit of the modular task trainer device. FIG. 3B depicts a side view of an exemplary base unit of the modular task trainer device. FIG. 3C depicts a back view of an exemplary base unit of the modular task trainer device.

FIG. 4 depicts a back-view of an exemplary base unit of the modular task trainer device with no module installed.

FIG. 5 depicts a back-view of an exemplary base unit of the modular task trainer device with a module installed.

FIG. 6 depicts a front view of an exemplary modular task trainer device, including the base unit and the plurality of modules.

FIG. 7A depicts an exemplary module for use with the modular task trainer device. FIG. 7B depicts an exemplary module for use with the modular task trainer device. FIG. 7C depicts an exemplary module for use with the modular task trainer device. FIG. 7D depicts an exemplary module for use with the modular task trainer device.

FIG. 8 depicts an exemplary base unit of the task trainer device and exemplary modules.

FIG. 9A depicts an exemplary modular task trainer device being used to simulate a tracheostomy procedure. FIG. 9B depicts an exemplary modular task trainer device being used to simulate a tracheostomy procedure.

FIG. 10 depicts the exemplary modular task trainer device being used with a laryngoscope.

### DETAILED DESCRIPTION

The following discussion omits or only briefly describes conventional features of medical simulation models or manikins that are apparent to those skilled in the art. Those of ordinary skill in the pertinent arts may thus recognize that other elements may be desirable and/or necessary to implement the devices, systems, and/or methods described herein. It is noted that various embodiments are described in detail with reference to the drawings. Reference to these various embodiments does not limit the scope of the claims attached hereto. Additionally, any embodiments set forth in this specification are intended to be non-limiting and merely set forth some of the many possible implementations for the appended claims. Further, particular features described herein can be used in combination with other described features in each of the various possible combinations and permutations. As such, it is understood that this detailed description is exemplary and explanatory only and is not restrictive of the broad inventive concepts upon which the embodiments disclosed herein are based.

Unless otherwise specifically defined herein, all terms are to be given their broadest reasonable interpretation. This includes meanings implied from the specification as well as meanings understood by those skilled in the art and/or as defined in dictionaries, treatises, etc.

It is noted that, as used in this specification and the appended claims, the singular forms “a,” “an,” and “the” include plural referents unless otherwise specified. The term “includes” and/or “including,” when used in this specification, specify the presence of stated features, elements, and/or components, but do not preclude the presence or addition of one or more other features, steps, operations, elements, components, and/or groups thereof.

Relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then-described or as shown in the drawing figure under discussion. These relative terms are for convenience of description and normally are not intended to require a particular orientation in actuality. Terms including “inwardly” versus “outwardly,” “longitudinal” versus “lateral,” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The phrases “operatively” or “operably connected” indicates such an attachment, coupling, or connection that allows the pertinent structures to operate as intended by virtue of that relationship.

Reference throughout the specification to “one embodiment,” “an embodiment,” or “some embodiments” means that a particular feature, structure, or characteristic described in connection with at least one embodiment of the subject matter is included in at least one embodiment of the subject matter disclosed. Thus, the appearance of the phrases “in one embodiment,” “in an embodiment,” or “in some embodiments” in various places throughout the specification is not necessarily referring to the same embodiment. Further, the particular features, structures, or characteristics of “one embodiment,” “an embodiment,” or “some embodiments” may be combined in any suitable manner with each other to form additional embodiments of such combinations. It is intended that embodiments of the disclosed subject matter cover modifications and variations thereof. Terms such as “first,” “second,” “third,” etc., merely identify one of a number of portions, components, steps, operations, functions, and/or points of reference as disclosed herein, and likewise to not necessarily limit embodiments of the present disclosure to any particular configuration or orientation.

Moreover, throughout this disclosure, various aspects can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range. As used herein, the term “about” in reference to a measurable value, such as an amount, a temporal duration, and the like, is meant to encompass the specified value or variations of plus or minus 20%, plus or minus 10%, plus or minus 5%, plus or minus 1%, and plus or minus 0.1% of the specified value, as such variations are appropriate and fit within the confines of a functional system.

The terms “proximal,” “distal,” “anterior,” “posterior,” “medial,” “lateral,” “superior,” and “inferior” are defined by their standard usage indicating a directional term of reference. For example, “proximal” refers to a position that is situated nearer to the center of a body or point of attachment or interest. In another example, “anterior” refers to the front of a body or structure, while “posterior” refers to the rear of a body or structure, in relation to a relative viewpoint. In another example, “medial” refers to the direction towards the midline of a body or structure, and “lateral” refers to the direction away from the midline of a body or structure. In some examples, “lateral” or “laterally” may refer to any sideways direction. In another example, “superior” refers to the top of a body or structure, while “inferior” refers to the bottom of a body or structure. It should be understood, however, that the directional term of reference may be interpreted within the context of a specific body or structure, such that a directional term referring to a location in the context of the reference body or structure may remain consistent as the orientation of the body or structure changes.

The terms “patient,” “subject,” “individual,” and the like are used interchangeably herein, and refer to any animal amenable to the systems, devices, and methods described herein. The patient, subject or individual may be a mammal, and in some instances, a human.

Aspects of the present disclosure relate to a modular tracheostomy task trainer device. The disclosed task trainer device is configured as a modular, anatomically accurate training and simulation device designed to replicate the human head and neck regions. In some embodiments, the disclosed task trainer device is configured for education and training for tracheostomy procedures, airway management, and/or suction procedures. In some embodiments, the disclosed task trainer device is configured to allow for the simulation of various emergency or complication scenarios that may arise during a tracheostomy procedure. In some embodiments, the modular design of the disclosed task trainer device offers flexibility, enabling users to customize the configuration of the device to simulate specific tracheostomy emergency or complication scenarios.

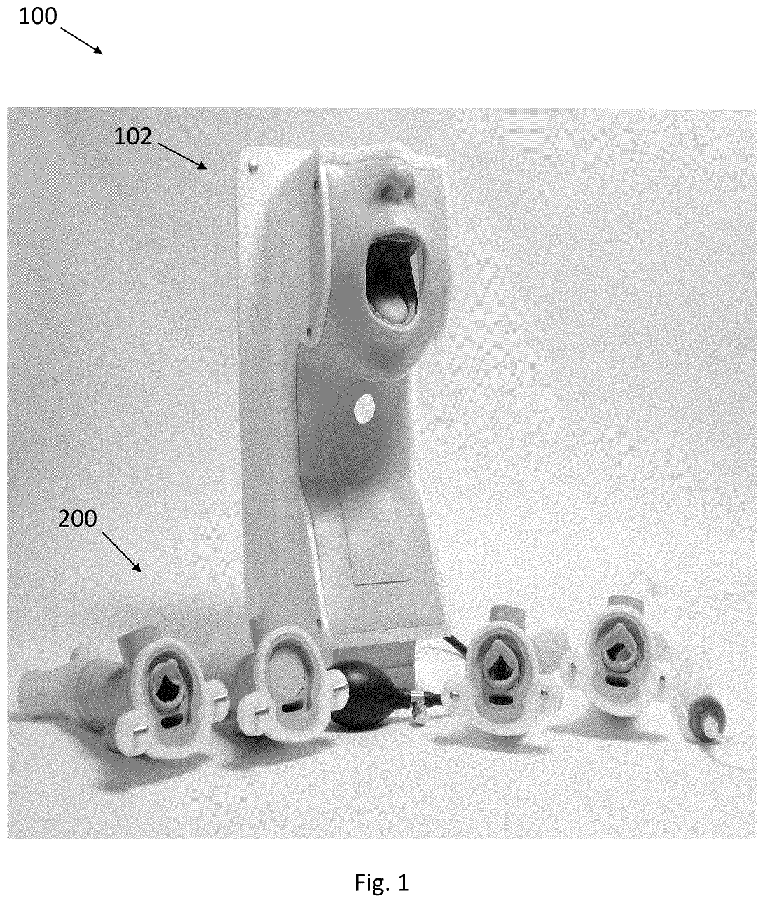

Referring now to FIG. 1, shown is a perspective view of an exemplary modular tracheostomy task trainer device 100 (hereinafter “device 100.”). The device 100 generally comprises a base unit 102 and a plurality of modules 200, wherein the base unit is configured to receive and releasably engage at least one module of the plurality of modules 200. In some embodiments, the base unit 102 is configured to replicate a human head and neck anatomy and the underlying tracheal system. In some embodiments, each module of the plurality of modules 200 replicates a tracheal and/or an esophageal system. In some embodiments, each module of the plurality of modules 200 is configured to enable the simulation of one or more tracheostomy complication scenarios and may comprise features specific to the particular complication scenario. In some embodiments, each module of the plurality of modules 200 is configured to be removably attached to the base unit 102. In some embodiments, the plurality of modules 200 are interchangeably attachable to the base unit 102 and easily swapped out based on the particular complication scenario to be simulated.

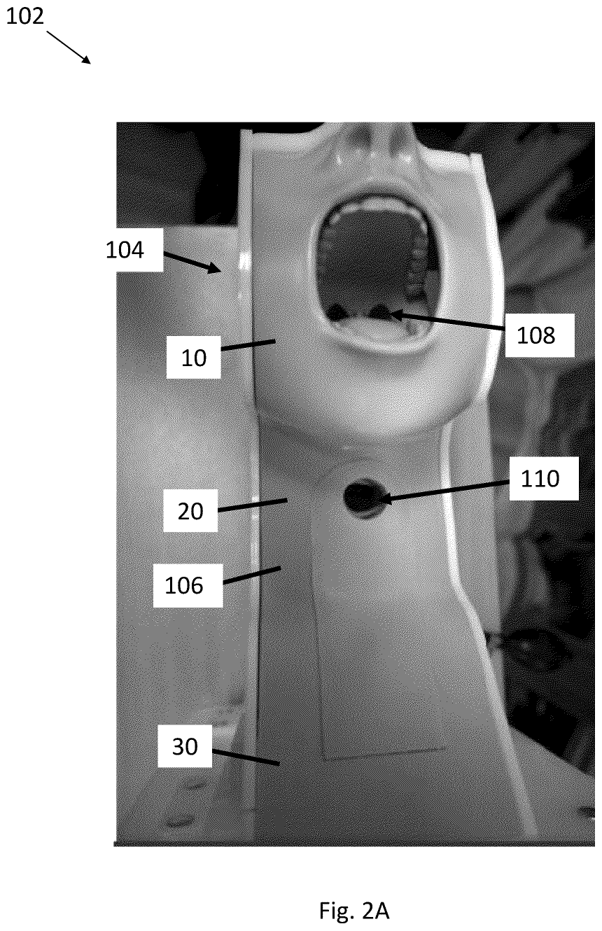

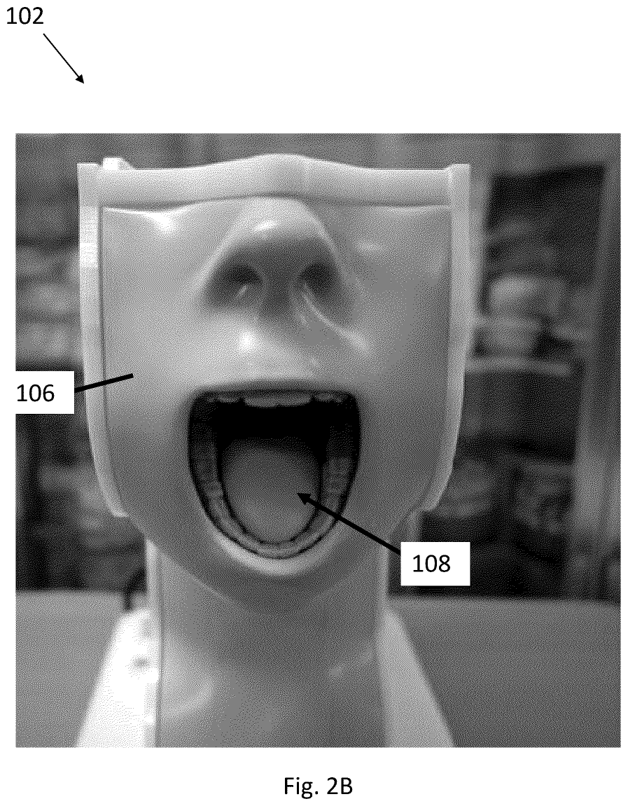

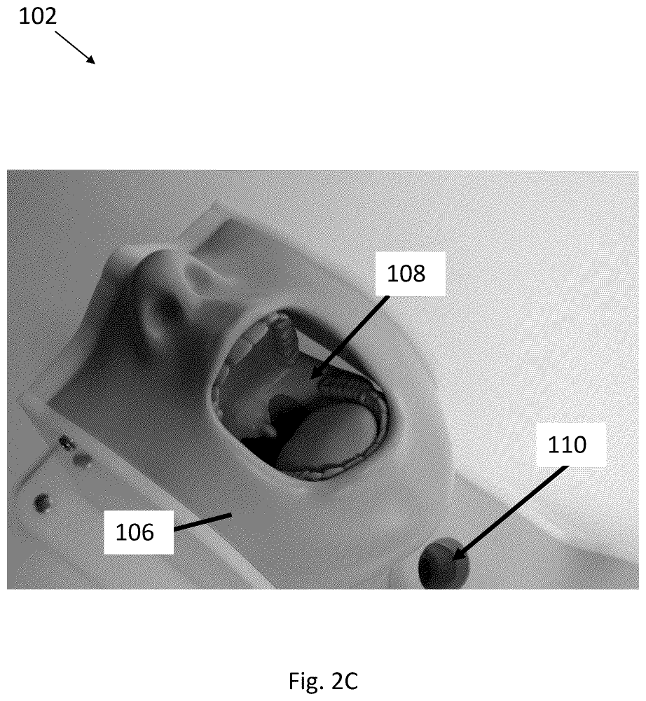

Referring now to FIG. 2A, shown is a front view of an exemplary base unit 102. The base unit 102 generally comprises a hollow body 104 defining an inner cavity therein and having a front surface 106. In some embodiments, the front surface 106 is configured to replicate at least a portion of the human body, such as a head, a neck, and optionally an upper chest area. In some embodiments, the front surface 106 is shaped to replicate the natural contours and anatomical features of the head and neck area. In some embodiments, the front surface 106 comprises a head portion 10, which may be configured to replicate the shape of a human head, wholly or partially, and which may include the face, jawline, and/or at least part of the cranial contour. In some embodiments, the front surface 106 comprises a neck portion 20, which may be configured to replicate the neck's curvature and musculature. In other embodiments, the front surface 106 further comprises an upper chest portion 30, which may be configured to replicate the clavicle and/or shoulder regions.

In some embodiments, the front surface 106 may be formed from rigid materials. In some embodiments, the front surface 106 may be formed from flexible materials. In some embodiments, the front surface 106 may be formed from a combination of rigid and flexible materials. In some embodiments, the front surface 106 may be formed from a rigid material layered with a flexible material. In some embodiments, the front surface 106 may comprise one or more layers of rigid or flexible materials. In some embodiments, the front surface 106 is formed from materials configured to provide a tactile response comparable to the human skin surface, and the underlying epidermal and endodermal layers. Examples of materials include, but not limited to, plastics, acrylic, metals, metal alloys, fiberglass, rubber, silicone, silicone rubber, cast urethane rubber, latex, polyethylene, polylactic acid (PLA), polyurethane, polyester, polyester resin, polyvinyl alcohol (PVA), polyether ether ketone (PEEK), acrylonitrile butadiene styrene (ABS), polycarbonate, polypropylene, carbon fibers, thermoplastic elastomers, urethane-based elastomers, resins, gels, foam or any combinations thereof.

In some embodiments, the front surface 106 includes an oral cavity 108, which may be a recessed area in the head portion 10, accessible via an oral opening or aperture in the front surface 106. In some embodiments, the oral cavity 108 may comprise structures configured to simulate one or more anatomical features of a human oral cavity including, but not limited to, the teeth, the tongue, the gums, the hard or soft palate, the uvula, and the like, or any combination thereof. In some embodiments, the front surface 106 includes a nasal cavity, which may be a recessed area in the head portion 10, separate from the oral cavity 108, and accessible via one or more nasal openings in the front surface 106. In some embodiments, the oral cavity 108 extends posteriorly into the base unit 102, the extension configured to replicate an upper airway of the throat (as described in more detail below). In some embodiments, the oral cavity 108 may comprise rigid materials, flexible materials, or combinations thereof. In some embodiments, the oral cavity 108 may comprise materials configured to replicate soft tissue textures in a human oral cavity.

In some embodiments, the front surface 106 includes a tracheal opening 110 in the neck portion 20, positioned where a tracheostomy incision would typically be placed. In some embodiments, the tracheal opening 110 provides access into the inner cavity of the base unit 102. In some embodiments, the tracheal opening 110 is a generally circular opening having a diameter sized to fit a conventional tracheostomy tube. In some embodiments, the tracheal opening 110 has a diameter ranging between about 2 mm and 20 mm, between about 3 mm and 19 mm, between about 4 mm and 18 mm, between about 5 mm and 17 mm, between about 6 mm and 16 mm, between about 7 mm and 15 mm, between about 8 mm and 14 mm, between about 9 mm and 13 mm, between about 10 mm and 12 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm. In some embodiments, the tracheal opening 110 may further comprise a removable cover or panel configured to seal the tracheal opening 110. In some embodiments, the front surface 104 comprises a puncturable and/or flexible area in the neck portion 20, such that the puncturable area may be incised and held open to form the tracheal opening 110. FIGS. 2B and 2C depict additional close-up views of the base unit 102 showing the oral cavity 108 and the tracheal opening 110.

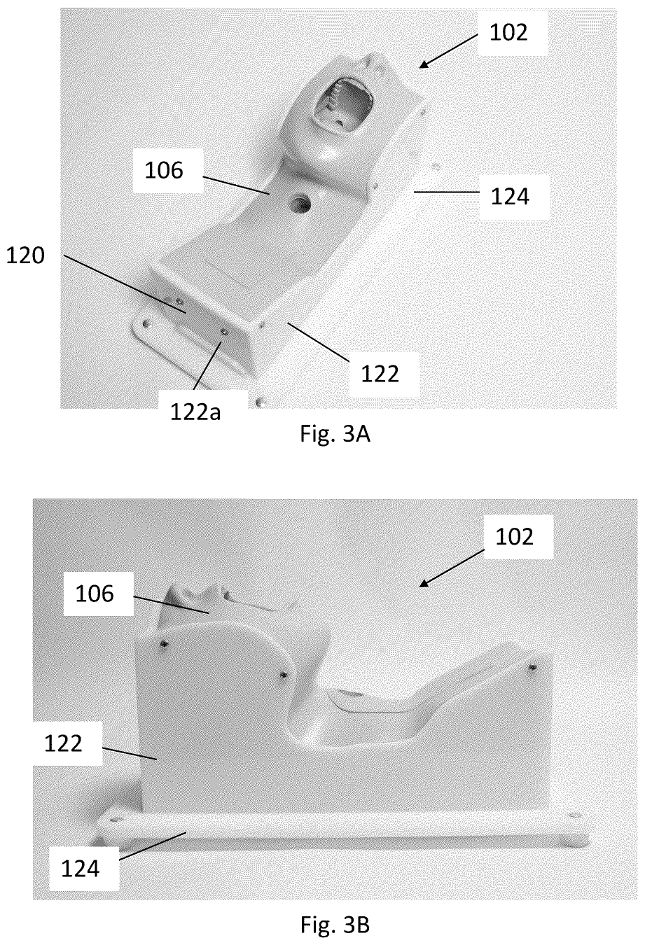

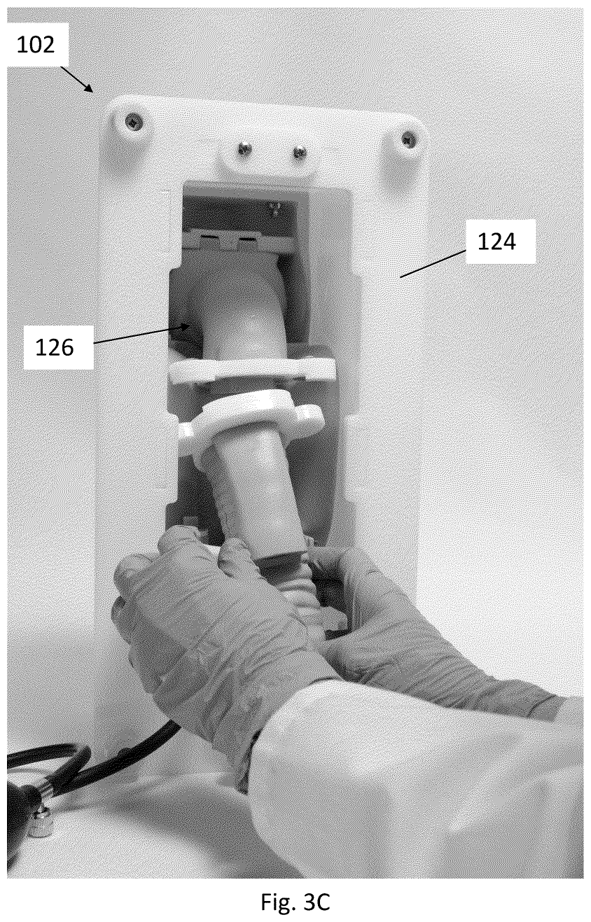

Referring now in detail to FIGS. 3A and 3B, shown are perspective views of the base unit 102. In some embodiments, the base unit 102 is housed in an outer casing 120. In some embodiments, the casing 120 comprises sidewalls 122 configured to enclose the lateral sides of the base unit 102 while leaving the front surface 104 exposed. In some embodiments, the sidewalls 122 are connected to a base 124, wherein the base 124 is substantially flat and configured to be positioned on top of a flat surface such that the simulation or training may be performed. In some embodiments, the base 124 may further comprise mounting supports or feet, configured to ensure the stability of the base unit 102. In some embodiments, the casing 120 may be attached to the base unit 102 via screws, bolts, clips, slot and tab mechanisms, pin and groove mechanisms, buttons, Velcro, adhesives, glues, and the like. In some embodiments, and referring now to FIG. 3C, the base 124 of casing 120 further comprises a posterior opening 126 configured to provide access to the inner cavity of the base unit 102. In some embodiments, the casing 120 may comprise a removable panel positioned on top of the opening 126 and fixedly and removably attached to the base 124 via screws, bolts, clips, magnets, snap-fit mechanisms, slot and tab mechanisms, pin and groove mechanisms, buttons, Velcro, adhesives, glues, and the like.

In some embodiments and referring back to FIG. 3A, at least a portion of the sidewalls 122 (such as sidewall portion 122a) may be hingedly connected to the back surface 124, such that the base 124 may remain positioned in a flat position, while the sidewalls 122 and the base unit 102 enclosed therein may be angled about an axis of rotation parallel to the length of the sidewall portion 122a. In other embodiments, the casing 120 may comprise a pivoting mount, or an adjustable tilt bracket configured to orient the base unit 102 to desired angles. In some embodiments, the base unit 102 may be utilized for simulation or training with the base unit 102 configured in a supine position, or in an inclined position at any desired angle ranging between 0° and 90°. In some embodiments, the base unit 102 may be configured to be oriented at a 15° incline, a 30° incline, a 45° incline, a 60° incline, a 75° incline, or any combinations thereof. In some embodiments, the casing 120 may be at least partially formed of substantially rigid materials including plastics, polymers, polylactic acid (PLA), polyurethane, polycarbonate, fiberglass, acrylic, metals, metal alloys, and the like, or any combinations thereof.

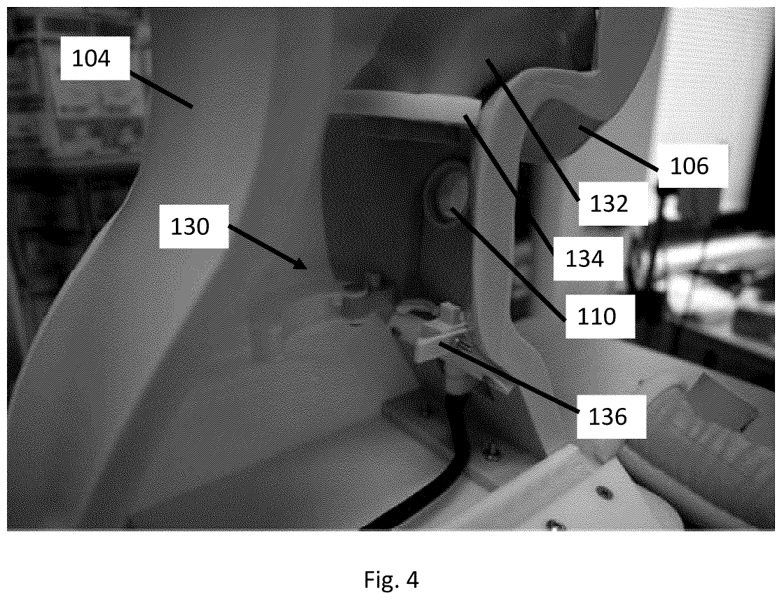

Referring now to FIG. 4, a back view of an exemplary base unit 102 is depicted. In some embodiments, the body 104 has an open posterior side configured to provide access to an inner cavity 130. In some embodiments, the inner cavity 130 is configured to receive at least one module of the plurality of modules 200. In some embodiments, the inner cavity 130 may comprise at least one attachment point configured to releasably connect to a module of the plurality of the modules 200. In some embodiments, the inner cavity 130 may comprise one or more structures configured to replicate one or more anatomical structures in the neck or throat as would be known by one of skill in the art.

In some embodiments, the base unit 102 comprises an upper airway portion 132 fluidly connected to and extending posteriorly from the oral cavity 108 on the front surface 106. In some embodiments, the upper airway portion 132 may further connect to a nasal cavity in the front surface 108. In some embodiments, the upper airway 132 is a continuous posterior extension of the oral cavity 108. In some embodiments, the upper airway 132 is generally configured as a tubular structure having an inner lumen, the inner lumen configured to provide an air passage through the base unit 102. In some embodiments, the upper airway 132 and the oral cavity 106 may be formed as a single component part and affixed to the inner surface of inner cavity 130 via adhesives, glues, snap-fit connections, slot and tab mechanisms, pin and groove mechanisms, magnets, Velcro, screws, bolts, and the like, or any combinations thereof.

In some embodiments, the oral cavity 106, the upper airway 132, and optionally the nasal cavity are configured to form a single continuous airway passage. In some embodiments, the oral cavity 106 and the upper airway 132 are configured to replicate the anatomical structures of the mouth and throat, and may be formed to comprise features corresponding to a tongue, teeth, uvula, gums, hard or soft palate, a pharynx, an oropharynx, a nasopharynx, a hypopharynx, a larynx, and epiglottis or any other anatomical structures in the upper throat as would be known to one of skill in the art. In some embodiments, the upper airway 132 comprises removable inserts positioned within the inner lumen of the upper airway 132. In some embodiments, the removable inserts are configured to replicate soft tissue textures, a mucosal lining, or pharyngeal and/or laryngeal features.

In some embodiments, the upper airway 132 may be formed from rigid materials, flexible materials, or combinations thereof. In some embodiments, the upper airway 132 may be formed from layers of different materials. In some embodiments, the inner surface of the upper airway 132 comprises materials configured to correspond to tissue textures within the throat. In some embodiments, the materials may be ridged, textured, patterned, lined, or otherwise modified to replicate soft tissues, mucosal linings, muscles, cartilage, cilia, and the like. In some embodiments, the upper airway 132 may be formed from materials including, but not limited to, plastics, acrylic, metals, metal alloys, fiberglass, rubber, silicone, silicone rubber, cast urethane rubber, latex, polyethylene, polylactic acid (PLA), polyurethane, polyester, polyester resin, polyvinyl alcohol (PVA), polyether ether ketone (PEEK), acrylonitrile butadiene styrene (ABS), polycarbonate, polypropylene, carbon fibers, thermoplastic elastomers, resins, gels, foam or any combinations thereof.

In some embodiments, the inner cavity 130 comprises a first attachment 134. In some embodiments, the first attachment 134 may be fixedly connected to an inferior end of the upper airway portion 132, or may be fixedly connected to the inner surface of the cavity 130. In some embodiments, the base unit 102 further comprises a second attachment 136 fixedly attached to the inner surface of the base unit 102 and positioned inferiorly to the first attachment 136. In some embodiments, the first and second attachments 134 and 136 are configured to receive and releasably engage at least one module of the plurality of modules 200.

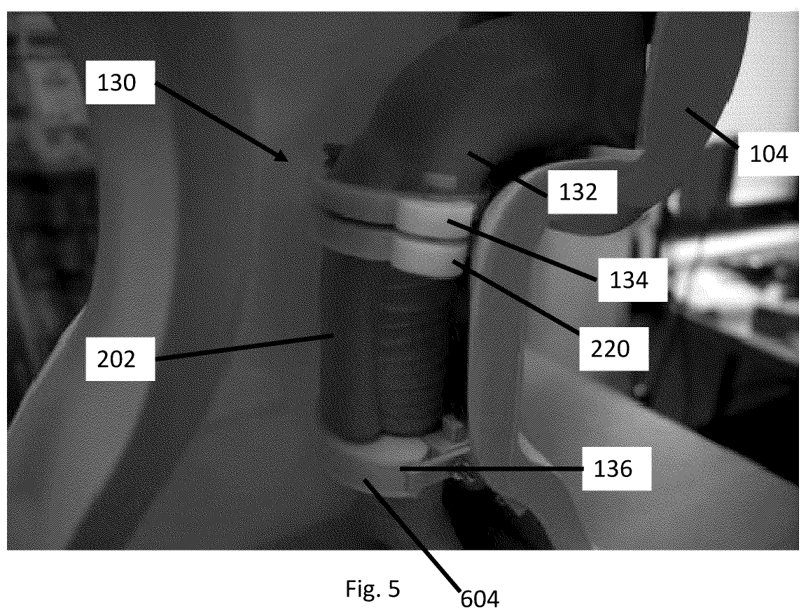

Referring now to FIG. 5, the modular task trainer device is shown with a module installed (exemplified in the depicted embodiment by module 202, but which may be any module of the plurality of modules 200). In some embodiments, at least one module is configured to be removably attached between the first and second attachments 134 and 136 of the base unit 102. In some embodiments, each module of the plurality of modules 200 comprises an upper attachment 220 configured to directly connect to the first attachment 134 of the base unit 102. In some embodiments, each module of the plurality of modules 200 may comprise a lower attachment configured to directly connect to the second attachment 136. In some embodiments, the junction between the upper attachment 220 of the module and the first attachment 134 of the base unit 102 may comprise a gasket, O-ring, or other seal configured to create an air-tight connection between the upper airway 132 and the module. In some embodiments, each module of the plurality of modules 200 further fluidly connects to the tracheal opening 110 via an opening in the module (e.g. opening 210 described below) such that the tracheal opening 110 on the front surface 106 provides access to an inner portion of the module, through which a tracheostomy procedure may be simulated. In some embodiments, the tracheal opening 110 may comprise a third attachment, configured to further fixedly secure the module to the base unit 102. In various embodiments, any of the attachments described above may comprise any type of fastener, for example clips, screws, magnets, slot and tab mechanisms, pin and groove mechanisms, adhesives, glues, Velcro, or any other suitable mechanism, or any combinations thereof.

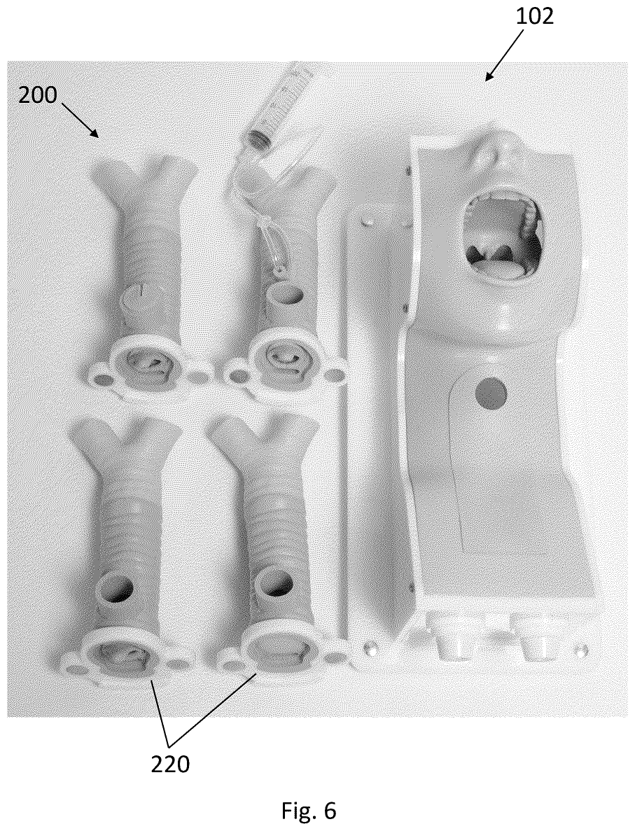

Referring now to FIG. 6, shown is a top-down view of an exemplary base unit 102 and the plurality of modules 200. In some embodiments, the plurality of modules 200 may comprise any number of modules. It should be appreciated that, while the plurality of modules 200 described herein are generally configured to replicate a tracheal and/or esophageal passage, the plurality of modules 200 may include additional releasably attachable modules configured to replicate one or more of: an oral cavity, a nasal cavity, a pharynx, a larynx, or any other structures of the throat.

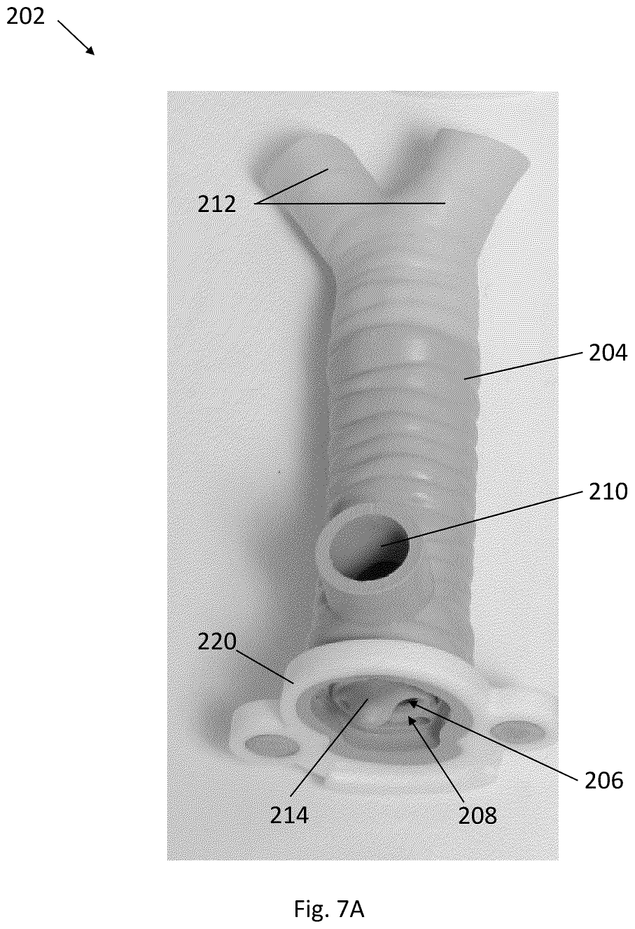

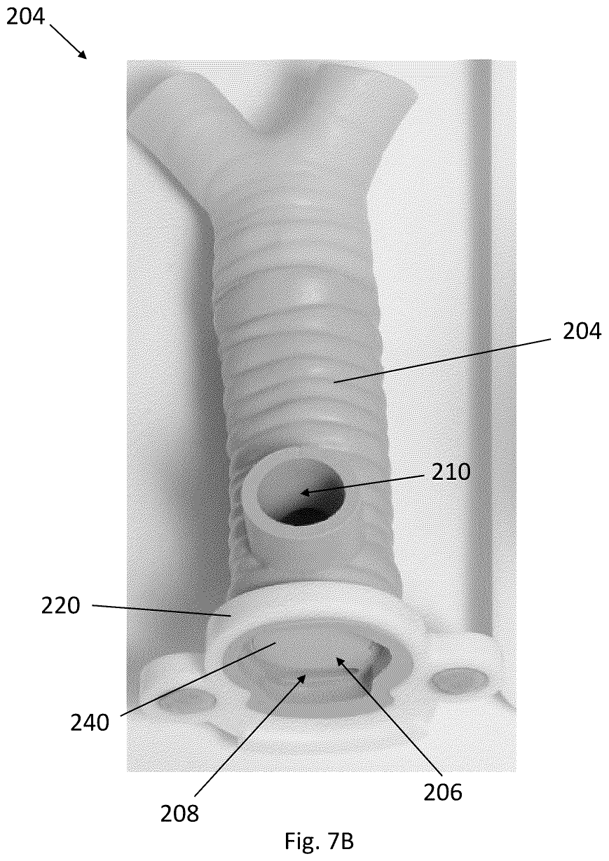

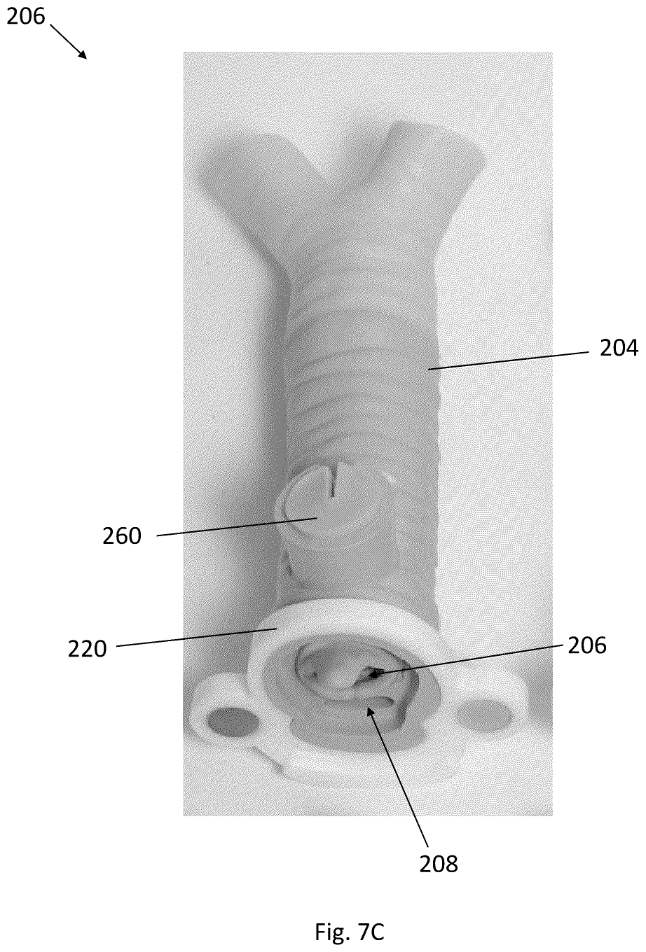

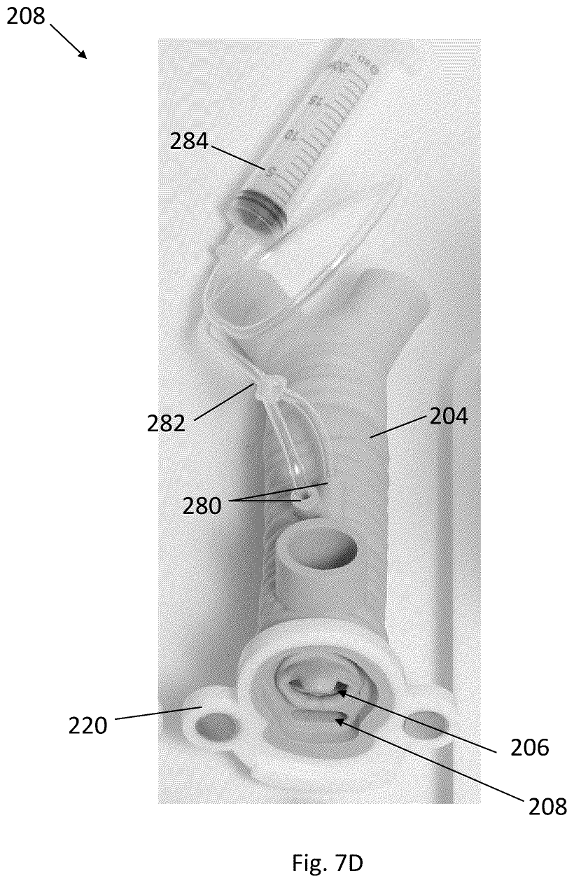

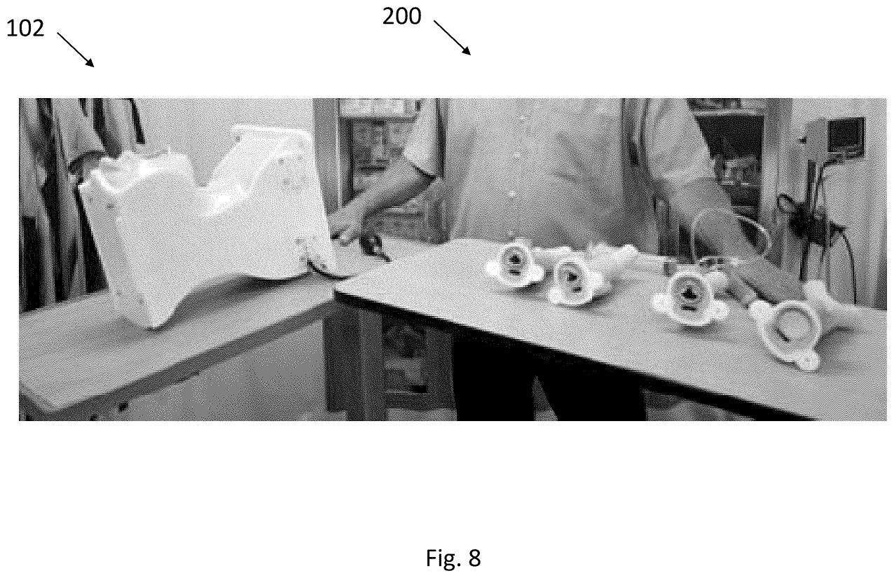

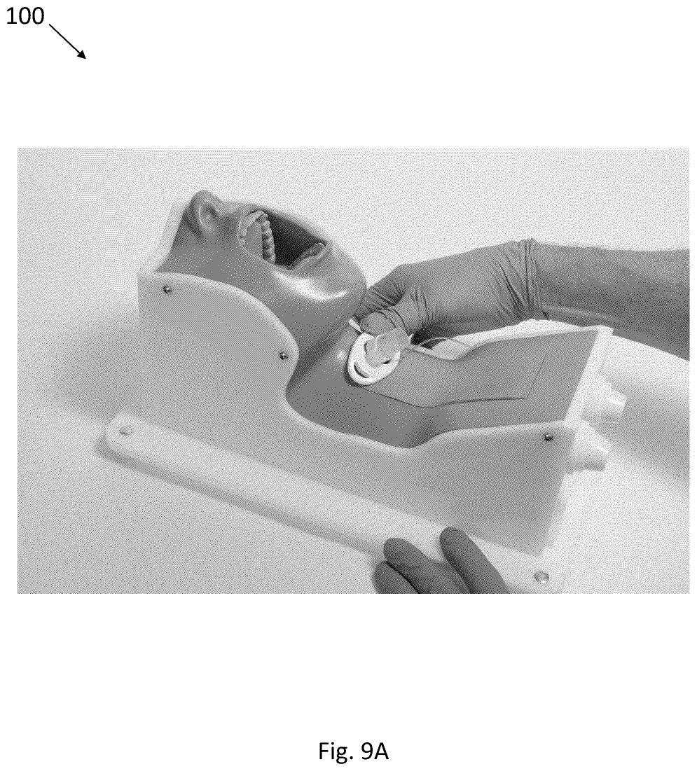

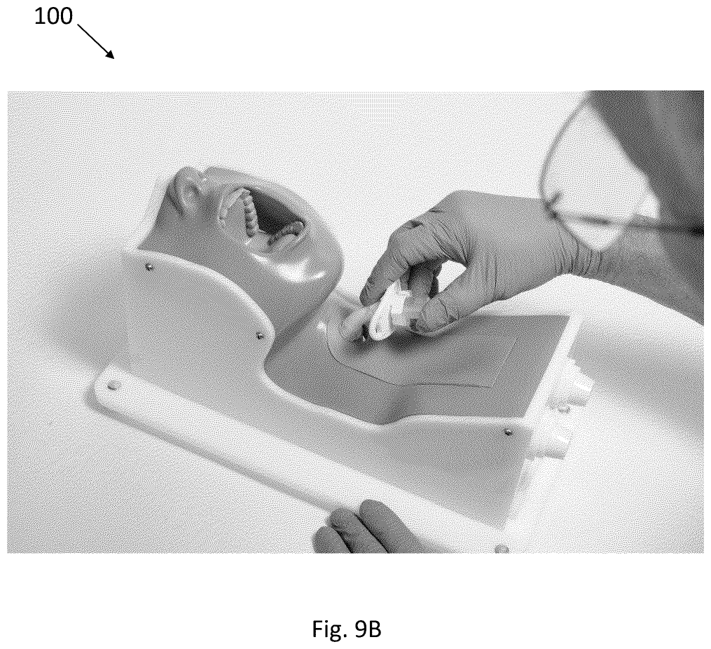

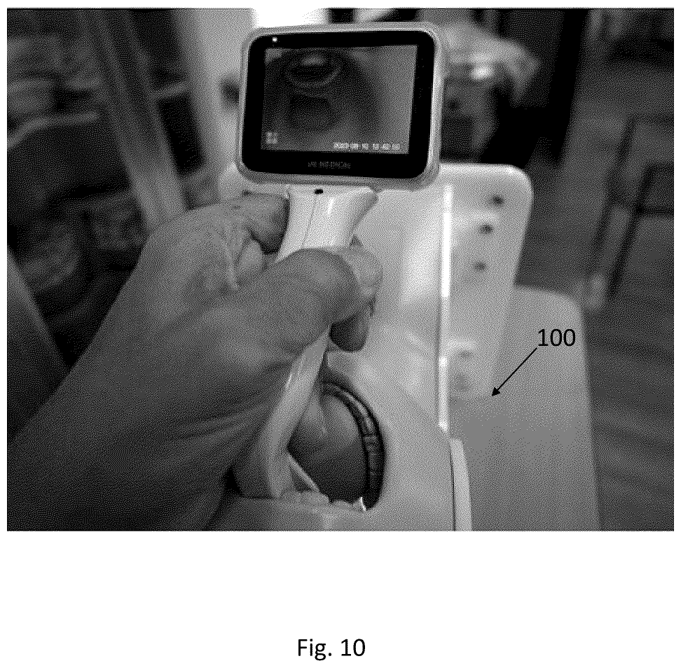

FIG. 7A illustrates the general features of each module of the plurality of modules 200, exemplified by the module 202. Generally, each module of the plurality of modules 200 is configured to replicate a tracheal passage and comprises at least one attachment 220 configured to releasably engage at least one attachment in the base unit 102. In some embodiments, each module of the plurality of modules 200 may comprise a tubular body 204 having a tracheal passage 206, and an esophageal passage 208, wherein the esophageal passage 208 is positioned posterior to the tracheal passage 206. In some embodiments, and when any module of the plurality of modules 200 is installed within the base unit 102, the inner lumen of the upper airway 132 is aligned with the tracheal and esophageal passages of the module 202 to form a continuous airway through the base unit 102 and to provide access to either the tracheal passage 206 or the esophageal passage 208 via the upper airway 132. In some embodiments, each module of the plurality of modules 200 further comprises an opening 210 in the body 204, the opening 210 providing access to the tracheal passage 206. In some embodiments, the opening 210 is positioned to align with the tracheal opening 110 of the base unit 102 when any module of the plurality of modules 200 is installed within the base unit 102. In some embodiments, the opening 210 may comprise a raised lip or flange configured to fit into the tracheal opening 110 and provide an airtight seal between the openings. In some embodiments, each module of the plurality of modules 200 may further comprise one or more bronchial passages 212 branching off inferiorly from the tracheal passage 206. In some embodiments, each module of the plurality of modules 200 further comprises an epiglottis 214 extending from the inner surface of the tracheal passage 206.

Each module of the plurality of modules 200 is generally shaped to replicate anatomical structures in the throat. For example, the body 204 may comprise flexible portions configured to replicate soft tissue, and rigid portions to replicate cartilage rings in the trachea. In some embodiments, the inner surface of each module of the plurality of modules 200 (or the inner surfaces of the tracheal or esophageal passages) may comprise materials configured to correspond to soft tissue textures within the throat. In some embodiments, the materials may be ridged, textured, patterned, lined, or otherwise customized to replicate one or more of: soft tissues, a mucosal lining, cilia, and the like, or any combination thereof. In some embodiments, materials may include, but not limited to, plastics, acrylic, metals, metal alloys, fiberglass, rubber, silicone, silicone rubber, cast urethane rubber, latex, polyethylene, polylactic acid (PLA), polyurethane, polyester, polyester resin, polyvinyl alcohol (PVA), polyether ether ketone (PEEK), acrylonitrile butadiene styrene (ABS), polycarbonate, polypropylene, carbon fibers, thermoplastic elastomers, resins, gels, foam or any combinations thereof. In some embodiments, each module of the plurality of modules 200 may be formed to comprise structures configured to replicate features such as the larynx, the vocal cords, the epiglottis, or the laryngeal cartilage.

In some embodiments, each module of the plurality of modules 200 may comprise one or more removable inserts configured to be positioned within the inner surface of the modules. In some embodiments, the one or more removable inserts are configured to simulate the soft tissue texture and/or features of the soft tissues of the trachea, a mucosal lining, the soft tissues of the esophagus, the epiglottis, the larynx, or any other anatomically relevant feature of the throat. In some embodiments, each module of the plurality of modules 200 may be configured to replicate features of a tracheostomy complication or scenario, such as obstructions or fluids within the tracheal passage.

Referring now to FIGS. 7A through 7D, shown are different exemplary configurations for each module of plurality of modules 200. It should be appreciated that the modules are not limited to the depicted configurations and may comprise additional modules comprising any desired additional features or structures. Further, the plurality of modules 200 may comprise additional modules having any combination of features of each module configuration described herein.

Referring now to FIG. 7A, shown is an exemplary module 202. In some embodiments, the module 202 is configured to replicate a normal tracheal anatomy. In some embodiments, the module 202 may be used to simulate a normal tracheostomy tube insertion with no complications. In some embodiments, the module 202 may be used to simulate the insertion or removal of a tracheostomy tube in a normal trachea. In some embodiments, the module 202 may be used to simulate a broken tracheostomy cuff. In such scenarios, the simulation may involve simulating the replacement of a tracheostomy tube. In some embodiments, the first module 202 may be used to simulate a block in the tracheostomy tube, wherein the tracheostomy tube comprises an inner obstruction. In such scenarios, the simulation may involve clearing the inner obstruction either through mechanical action or via suctioning, replacing the inner cannula of the tracheostomy tube, replacing the entire tracheostomy tube, or placing an endotracheal tube through the mouth. In some embodiments, the inner obstruction may be formed from any substantially rigid, flexible, or gelatinous material and may be configured to replicate a blood clot, a mucus plug, and the like. In some embodiments, the inner obstruction may be positioned within the tracheostomy tube prior to the start of the simulation exercise. In some embodiments, the inner obstruction may be fixedly attached to the tracheostomy tube via adhesives, glues, and the like. In some embodiments, the module 202 may be used to simulate the placement and removal of an endotracheal tube or to simulate airway management techniques.

Referring now to FIG. 7B, shown is an exemplary module 204. The module 204 may comprise a tracheal obstruction 240 configured to replicate an obstruction or blockage in the tracheal passage 206. In some embodiments, the tracheal obstruction 240 may be configured to completely or partially obstruct the tracheal passage 206. In some embodiments, the obstruction 240 may be formed from rigid or flexible materials, and may be configured to replicate soft tissues, blood clots, mucus plugs, and the like. In some embodiments, the obstruction 240 may be configured to be removable, or may be fixedly attached to the tracheal passage 206. In some embodiments, the module 204 may be used to simulate the trachea of a patient having undergone a total laryngectomy. In total laryngectomies, the entire larynx (voice box) is removed, and there is no longer a connection between the mouth and the trachea. In some embodiments, the module 204 may be configured in any way to fully or partially obstruct the connection between the trachea and the mouth, such that it is not possible to insert an endotracheal tube from the mouth.

Referring now to FIG. 7C, shown is an exemplary module 206. In some embodiments, the module 206 may comprise an obstruction 260 in the opening 210 of the module. In some embodiments, the tracheal obstruction 260 may be configured to completely or partially obstruct the opening 210, such that the tracheal passage is no longer accessible from the opening 210. In some embodiments, the obstruction 260 may be formed from rigid or flexible materials, and may be configured to replicate soft tissues, blood clots, mucus plugs, and the like. In some embodiments, the obstruction 260 may be configured to be removable, or may be fixedly attached to the opening 210. In some embodiments, the module 206 may be used to simulate the displacement of the tracheostomy tube in a false passage.

Referring now to FIG. 7D, shown is an exemplary module 208. In some embodiments, the module 208 comprises one or more inlets or ports 280 fluidly connected to the tracheal passage 206. In some embodiments, one or more conduits 282 may be fluidly connected between the one or more inlets 280 and at least one fluid supply 284. The fluid may be any fluid simulating blood, mucus, or any other fluid that may be found in the trachea. For example, the fluid may be configured to simulate blood and may comprise one or more materials selected from the group consisting of: artificial blood, water, dyed water, red dyed water, and the like. In other cases, the fluid may simulate mucus and may comprise any fluid having a viscosity corresponding to that of mucus. In some embodiments, the fluid may be provided by at least one fluid supply 284 fluidly connected to the one or more inlets 280 via the one or more conduits 282. In some embodiments, the one or more conduits 282 are formed from convention tubing materials such as polyvinyl chloride (PVC), polyurethane, or silicone. In some embodiments, the one or more conduits may be housed within the module and/or the base unit 102. In some embodiments, the fluid supply 284 may be housed within the base unit 102. In some embodiments, the at least one fluid supply 284 may comprise an external reservoir (e.g. a syringe). In some embodiments, the at least one fluid supply 284 may comprise a pump and/or valves adapted to control the rate and/or pressure of fluid simulant flow. In some embodiments, the fluid flow may be controlled by any suitable mechanism (e.g. the mechanical action of pushing down the plunger of a syringe.) In some embodiments, the module 208 may be used to simulate scenarios in which excessive fluid accumulates in the tracheal passage (e.g. complications with massive bleeding, or excessive mucus). In some embodiments, the module 204 may be used to simulate the management of excessive fluid via methods including suctioning to clear the airway, applying direct pressure to stop bleeding, or inflation of a cuff of the tracheostomy tube to tamponade a bleeding vessel.

In some embodiments, each module of the plurality of modules 200 may be fluidly connected to one or more lung bags. In some embodiments, one or more lung bags may be fluidly connected to each module of the plurality of modules 200 via the one or more bronchial passages of each module. In some embodiments, the one or more lung bags may be inflatable bags or sacs comprising a flexible, distensible, or elastic material. In some embodiments, the one or more lung bags may be configured to simulate the inflation and deflation of the lungs, thereby providing visual feedback during ventilation simulation. In some embodiments, the one or more lung bags may be configured to provide positive aspiration. In some embodiments, the oral cavity 108 of the base unit 102 may be adapted for fluid connection to a bag valve mask, or any other manual resuscitator device. In some embodiments, the base unit 102 and each module of the plurality of modules 200 may be configured for use with a laryngoscope, or any other imaging device as would be known to one of skill in the art.

Aspects of the present disclosure relate to dimensions for the device 100. In some embodiments, the device 100 is sized to be portable for easy transport. In some embodiments, the base unit 102 and the plurality of modules 200 may be dimensioned to simulate the head, neck, and torso area of an adult male or an adult female. In some embodiments, the base unit 102 and the plurality of modules 200 may be dimensioned to simulate the head, neck, and torso area of a child aged between 0 and 12 years old. In some embodiments, the length of the base unit may range between about 10 cm and 50 cm, between about 15 cm and 45 cm, between about 20 cm and 40 cm, or between about 25 cm and 30 cm. In some embodiments, the average width of the base unit 102 may range between 10 cm and 50 cm, between about 15 cm and 45 cm, between about 20 cm and 40 cm, or between about 25 cm and 30 cm. In some embodiments, each module of the plurality of modules 200 may have a length ranging between about 3 cm and 15 cm, between about 4 cm and 14 cm, between about 5 cm and 13 cm, between about 6 cm and 12 cm, between about 7 cm and 11 cm, or between about 8 cm and 10 cm. In some embodiments, each module of the plurality of modules 200 may have an average width or diameter ranging between about 2 mm and 25 mm, between about 5 mm and 20 mm, or between about 10 mm and 15 mm. It should be understood that while the present disclosure is described herein as configured to replicate a human head and neck anatomy, it is possible to configure the base unit 102 and the plurality of modules 200 to represent any mammalian animal species with which veterinarian tracheal intubation and procedures may be practiced.

Aspects of the present disclosure relate to methods of manufacturing the device 100. In some embodiments, the base unit 102 and any components thereof, and the set of modules 200, may be formed via injection molding, cast molding, 3D printing, or any other methods known to one of skill in the art, and any combinations thereof. In some embodiments, the base unit 102 may be formed via the assembly of component parts or may be formed as a single unitary piece. In some embodiments, the base unit 102 and any components thereof and the plurality of modules 200 may be formed utilizing computer-generated models. In some embodiments, the base unit 102 and the plurality of modules 200 may be molded to have sufficient anatomical detail to provide an anatomically accurate representation of the head, neck, throat, and any structures or features thereof. In some embodiments, the molding materials may be chosen to mimic human tissue properties, and be durable, strain resistant, and easy to clean.

Aspects of the present disclosure relate to training exercises or simulations that may be performed with the device 100. In some embodiments, the device 100 may be utilized for simulating tracheostomy tube insertion procedures, tracheostomy tube maintenance procedures, endotracheal tube insertion procedures, nasal intubation procedures, laryngoscope insertion, visualization procedures, airway management techniques, suction, extubation procedures, resuscitation techniques such as the use of manual resuscitation devices, and the like, or any combinations thereof. In some embodiments, the device 100 may be used to simulate tracheostomy procedures with a complication or in an emergency scenario, including but not limited to, a broken tracheostomy cuff, a blocked tracheostomy tube, displacement of the tracheostomy tube, cases with patients having undergone a total laryngectomy, or complications causing massive bleeding or excessive mucus in the trachea.

Aspects of the present disclosure relate to a tracheostomy training kit, comprising any modular tracheostomy task trainer described above (e.g. device 100) comprising the base unit 102 and a plurality of interchangeable modules (e.g. the plurality of modules 200). In some embodiments, the training kit may comprise a tracheostomy tube. In some embodiments, the tracheostomy tube may be provided in a set of different diameters configured to simulate tracheostomy procedures on devices 100 having a plurality of different sizes. In some embodiments, the tracheostomy tube may be cuffed tracheostomy tube, a cuffless tracheostomy tube, a fenestrated tracheostomy tube, a non-fenestrated tracheostomy tube, a single-cannula tracheostomy tube, a double-cannula tracheostomy tube, tracheostomy tubes having integrated suction, or any other tracheostomy tube known to one of skill in the art. In some embodiments, the training kit may further comprise one or more endotracheal tubes, one or more bag valve masks, one or more lung bags, or any other resuscitation device known to one of skill in the art. In some embodiments, the training kit may comprise a laryngoscope, or any other conventional imaging device known to one of skill in the art. In some embodiments, the training kit may comprise one or more tools such as needles, scalpels, cannulas, catheters, guide wires, pumps, suctions, or any other surgical instruments. In some embodiments, the training kit may further comprise any of: a fluid supply, a blood simulant, a blood simulant supply, a mucus simulant, a mucus simulant supply, one or more valves, one or more conduits, one or more connectors, and one or more syringes. In some embodiments, the training kit may further comprise a plurality of devices 100, each device 100 being differently sized to correspond to roughly correspond to an adult male, an adult female, or a child aged between the ages of 0 to 12 years old. It should be appreciated that the tracheostomy tubes, endotracheal tubes, bag valve masks, lung bags, or any other component of the training kit may be provided in different dimensions suited to the size of each device 100.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Educational Program for Pilot Testing

A pilot test for an education program with graduating medical residents was conducted. All participants went through an individualized training session using the task trainer. Pre and post training session surveys were completed by the participants and the training session was recorded. Five distinct emergencies were presented using the modular task trainer. Faculty led debriefings (educational discussion) occurred at the completion of each case. Validated checklists were used during subsequent video reviews to document correct and incorrect management decisions. Learners returned 3-4 months after their initial experience to go through the same cases to help determine if there was improvement in management of various emergencies that persisted for the 3-4 month period after the educational intervention.

A modular tracheostomy trainer was designed that could be used for both formative educational sessions and summative assessments. The model was piloted with individualized formative educational sessions on a convenience sample of graduating anesthesiology residents and critical care fellows. Each learner managed five tracheostomy emergencies involving a patient with hypoxic respiratory failure and respiratory distress. A bedside nurse provided relevant history when asked. Evolving vital signs were displayed on a bedside monitor. An educational debrief was held after each scenario.

Pre-session surveys were used to collect data on demographics, self-confidence in managing tracheostomy emergencies, and prior education and experience in managing these emergencies. Post-session surveys were used to assess the usability and realism of the task trainer, and the educational value of this new curriculum. Respondents rated their level of agreement with survey questions on a 5-point Likert scale (1=strongly disagree, 3=neutral, 5=strongly agree). Each individualized formative session was recorded and subsequently graded using checklists created by experts in both airway management and simulation-based education. A modified Delphi process was used for checklist development. Each resident was asked to manage 5 distinct tracheostomy emergencies—a focused debriefing occurred after completion of each case. Residents returned 3-4 months after their initial session for a summative assessment using the same cases.

Results from the pre-intervention survey are presented in Table 1.

TABLE 1
Pre-Intervention Survey

| | ANESTH | CCM |
|---|---|---|
| | (n = 8) | (n = 7) |
| | (mean/IQR) | |
| I feel comfortable with my ability to evaluate | 2.4/2-3 | 3.2/3-4 |
| and manage tracheostomy emergencies | | |
| I believe I have received adequate education | 2.5/2-3 | 3.3/3-4 |
| during my training program to evaluate and | | |
| manage tracheostomy related emergencies | | |
| I believe that didactic presentations adequately | 2.5/2-3 | 2.7/3-4 |
| prepared me to manage tracheostomy related | | |
| emergencies. | | |

Results from the post-intervention survey are presented in Table 2.

TABLE 2
Post-Intervention Survey

| | |
|---|---|
| ANESTH | CCM |
| (n = 8) | (n = 7) |

(mean/IQR)

Usability

| | | |
|---|---|---|
| I found that the task-trainer allowed me to perform | 5/5-5 | 5/5-5 |
| the hands-on tasks required to manage various trach | | |
| emergencies | | |
| I found that the task trainer was a useful tool to | 5/5-5 | 4.7/5-5 |
| improve my understanding and ability to assess and | | |
| manage various tracheostomy emergencies | | |

Realism

| | | |
|---|---|---|
| The task trainer realistically simulated the relevant | 4.9/5-5 | 4.6/4-5 |
| anatomy | | |
| The soft-tissue behavior and tactile sensation | 4.7/4-5 | 4.4/3-5 |
| associated with management of the various trach | | |
| emergencies was realistic | | |
| I found the task trainer realistically simulated trach | 4.9/5-5 | 4.6/4-5 |
| emergencies | | |

Educational Effects

| | | |
|---|---|---|
| I believe that this task trainer would be a useful | 5/5-5 | 5/5-5 |
| addition for the training of healthcare professionals | | |
| in the evaluation and management of trach | | |
| emergencies | | |
| I would recommend the use of this simulation | 5/5-5 | 5/5-5 |
| experience as a teaching tool for the assessment and | | |
| management of trach emergencies | | |
| I believe that after this simulation experience, my | 5/5-5 | 4.8/5-5 |
| self-confidence in my ability to evaluate and manage | | |
| trach complications has increased | | |
| I believe that after this simulation experience, my | 5/5-5 | 4.8/5-5 |
| skills for evaluating and managing various trach | | |
| emergencies has increased | | |

Preliminary data comparing positive change in checklist scores between session 1 and session 2 for 21 residents who have completed both sessions are presented in Table 3.

TABLE 3

| | Wilcoxon signed |
|---|---|
| | rank test |
| Case 1. Cuff leak due to ruptured cuff (chronic trach) | p = 0.008 |
| Case 2. Plugged inner cannula (fresh trach) | p < 0.001 |
| Case 3. Displaced trach total laryngectomy patient | p < 0.001 |
| Case 4. Displacement into a false passage (fresh trach) | p = 0.332 |
| Case 5. Massive bleeding from a T1 fistula (chronic | p = 0.003 |
| trach) | |

The pre-intervention survey documented widespread agreement in the inadequacy of prior tracheostomy emergency management training. This was likely a key factor contributing to a lack of self-reported comfort with participant's ability to respond effectively to these life-threatening emergencies. In the post-intervention survey, participants rated the realism and usability of the task trainer very highly, and when combined with debriefing of each scenario, the program was deemed to be a useful addition to healthcare professional training. In addition, the program not only increased participants' self-confidence in their ability to evaluate and manage these emergencies but also improved their skills to do so.

The use of a novel modular tracheostomy task trainer combined with the use of common and/or life-threatening complication scenarios with expert debriefing was associated with excellent educational effects and was recommended by all participants to be added to their training curricula.

Widespread education of healthcare providers in the evaluation and management of common and/or life-threatening tracheostomy-related emergencies may lead to enhanced patient safety and improved hospital outcomes for these at-risk patients.

### REFERENCES

1. Cook, T M et al. “Major complications of airway management in the UK: results of the Fourth National Audit Project of the Royal College of Anaesthetists and the Difficult Airway Society. Part 2: intensive care and emergency departments.” British journal of anaesthesia vol. 106, 5 (2011): 632-42.
2. Baker, P. A et al. (2011) “Education in airway management.” Anaesthesia, 66:101-111.
3. Roux D et al. Crit Care Med 2014. “Acquiring Procedural Skills in ICUs: A Prospective Multicenter Study”. 42(4): 886-895

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A modular tracheostomy task trainer device comprising:
a base unit comprising at least one attachment point; and
at least one module comprising an attachment point configured to removably attach to the at least one attachment point of the base unit.

2. The device of claim 1, further comprising a plurality of modules, wherein each module of the plurality of modules are interchangeable.

3. The device of claim 1, wherein the base unit comprises a body having a cavity therein, the cavity configured to receive the at least one module.

1. The device of claim 1, wherein the base unit is shaped to replicate the head and neck anatomy and comprises an anatomical feature selected from a mouth or a nose.

2. The device of claim 1, wherein the base unit comprises an oral cavity fluidly connected to an upper air passage.

3. The device of claim 5, wherein the upper air passage comprises structures configured to replicate one or more of a pharynx, a nasopharynx, an oropharynx, a hypopharynx, a larynx, or an epiglottis.

4. The device of claim 5, wherein the at least one module comprises a tracheal passage and an esophageal passage, wherein each passage is fluidly connectable to the upper air passage.

5. The device of claim 7, wherein the at least one module comprises an opening in the tracheal passage, the opening sized to fit a tracheostomy tube.

6. The device of claim 8, wherein the at least one module replicates a normal trachea.

7. The device of claim 8, wherein the at least one module comprises an obstruction in the tracheal passage, wherein the obstruction may be configured to fully or partially obstruct the tracheal passage.

8. The device of claim 8, wherein the at least one module comprises an obstruction in the opening in the tracheal passage, configured to prevent the insertion of a tracheostomy tube into the opening.

9. The device of claim 8, wherein the at least one module comprises one or more inlets in the tracheal passage fluidly connected to a fluid supply, wherein the one or more inlets are configured to introduce one or more fluids to the tracheal passage.

10. The device of claim 12, wherein the one or more fluids may be a blood simulant or a mucus simulant.

11. The device of claim 1, wherein the base unit and the at least one module are formed of materials configured to replicate soft tissue textures, muscle, cartilage, mucosal lining, or cilia.

12. The device of claim 14, wherein the materials are selected from the group consisting of: plastics, acrylic, metals, metal alloys, fiberglass, rubber, silicone, silicone rubber, cast urethane rubber, latex, polyethylene, polylactic acid (PLA), polyurethane, polyester, polyester resin, polyvinyl alcohol (PVA), polyether ether ketone (PEEK), acrylonitrile butadiene styrene (ABS), polycarbonate, polypropylene, carbon fibers, thermoplastic elastomers, resins, gels, foam, and any combinations thereof.

13. The device of claim 1, wherein the base unit comprises at least a first and a second attachment point.

14. The device of claim 16, wherein the at least one module comprises at least a first and a second attachment point, configured to removably attach to the first and second attachment points of the base unit, respectively.

15. The device of claim 1, further comprising a seal between the at least one attachment point of the base unit and the at least one attachment point of the at least one module.

16. The device of claim 1, wherein the base unit and the at least one module are sized to replicate the head and neck anatomy of an average adult.

17. The device of claim 1, wherein the base unit and the at least one module are sized to replicate the head and anatomy of a child between the ages of 0 and 12 years old.

18. The device of claim 1, wherein the device is configured to simulate one or more of: tracheostomy tube insertion procedures, tracheostomy tube maintenance procedures, endotracheal tube insertion procedures, nasal intubation procedures, extubation procedures, laryngoscope insertion, visualization procedures, airway management techniques, suction, resuscitation techniques, and any combinations thereof.

19. The device of claim 1, wherein the base unit and the at least one module are formed via injection molding, cast molding, or 3D printing.

20. A training kit for tracheostomy procedures, comprising:
the modular tracheostomy task trainer device of claim 1, and
one or more tracheostomy tubes, one or more endotracheal tubes, one or more resuscitator devices, one or more lung bags, one or more suction devices, one or more conduits, one or more pumps, one or more valves, a blood simulant, a blood simulant supply, a mucus simulant, a mucus simulant supply, or any combinations thereof.

21. The training kit of claim 23, further comprising one or more modular tracheostomy task trainer devices, each device being sized to simulate tracheostomy procedures on an average adult, or a child between the ages of 0 and 12 years old.
